(19) United States
(12) Patent Application Publication
(10) Pub. No.: US 2022/0400936 A1
(43) Pub. Date: Dec. 22, 2022

(54) ENDOSCOPIC AND FLUID MANAGEMENT SYSTEMS HAVING AN ELECTRONICALLY ADJUSTABLE ORIFICE

(71) Applicant: BOSTON SCIENTIFIC SCIMED, INC., Maple Grove, MN (US)

(72) Inventors: Troy Velazquez, Salem, NH (US); Damien Fitzgerald, Tipperary (IE)

(73) Assignee: BOSTON SCIENTIFIC SCIMED, INC., Maple Grove, MN (US)

(21) Appl. No.: 17/841,310

(22) Filed: Jun. 15, 2022

Related U.S. Application Data
(60) Provisional application No. 63/211,310, filed on Jun. 16, 2021.

(51) Int. Cl. A61B 1/015; A61B 1/00

(52) U.S. Cl. CPC A61B 1/015; A61B 1/00006; A61B 1/00057

(57) ABSTRACT

An endoscopic system may include an endoscope including a handle and an elongate shaft extending distally from the handle, wherein the handle includes an inflow port in fluid communication with the elongate shaft, the inflow port being configured to fluidly connect to a fluid inflow line, an electronically adjustable orifice associated with the inflow port, and control circuitry for receiving a signal of a current size of the electronically adjustable orifice and/or for sending a signal to change a size of the electronically adjustable orifice. The system may include a first pressure sensor disposed upstream of the electronically adjustable orifice and a second pressure sensor disposed downstream of the electronically adjustable orifice.

### CROSS REFERENCE TO RELATED APPLICATIONS

This application claims the benefit of and priority to U.S. Provisional Patent Application Serial No. 63/211,310 filed on Jun. 16, 2021, the disclosure of which is incorporated herein by reference.

### TECHNICAL FIELD

The disclosure is directed to an endoscopic system and/or a fluid management system for endoscopic systems. More particularly, the disclosure is directed to an electronically controlled adjustable orifice in an endoscopic system and/or a fluid management system.

### BACKGROUND

Flexible ureteroscopy (fURS), gynecology, and other endoscopic procedures may require the circulation of fluid for various reasons. Surgeons today deliver the fluid in various ways such as, for example, by hanging a fluid bag and using gravity to deliver the fluid, filling a syringe and manually injecting the fluid, or using a peristaltic pump to deliver fluid from a fluid source at a selected pressure or flow rate via a fluid management system. Some systems may include a stopcock permitting manual manipulation of the fluid delivery and/or the flow rate by the operator. Of the known medical devices, systems, and methods, each has certain advantages and disadvantages. For example, existing systems may offer limited control over pressure and/or flow rate. In some cases, manual closure of a stopcock may work against the fluid management system by terminating flow and/or causing pressure build-up within the fluid management system. There is an ongoing need to provide alternative endoscopic and/or fluid management systems.

### SUMMARY

In a first example, an endoscopic system may comprise an endoscope including a handle and an elongate shaft extending distally from the handle, wherein the handle includes an inflow port in fluid communication with the elongate shaft, the inflow port being configured to fluidly connect to a fluid inflow line, an electronically adjustable orifice associated with the inflow port, and control circuitry for receiving a signal of a current size of the electronically adjustable orifice and/or for sending a signal to change a size of the electronically adjustable orifice.

In addition or alternatively to any example described herein, the endoscopic system may further include a first pressure sensor disposed upstream of the electronically adjustable orifice and a second pressure sensor disposed downstream of the electronically adjustable orifice and upstream of the elongate shaft.

In addition or alternatively to any example described herein, the control circuity is configured to calculate an approximate current flow rate of a fluid passing through the electronically adjustable orifice based on the current size of the electronically adjustable orifice, a first fluid pressure measured by the first pressure sensor, and a second fluid pressure measured by the second pressure sensor.

In addition or alternatively to any example described herein, the endoscopic system may further comprise an inflow pump configured to pump a fluid through the fluid inflow line to the inflow port, wherein the control circuitry is configured to calculate an approximate current flow rate of a fluid passing through the electronically adjustable orifice based on the current size of the electronically adjustable orifice and a system pressure of the fluid measured between the inflow pump and the electronically adjustable orifice.

In addition or alternatively to any example described herein, the electronically adjustable orifice includes an adjustable iris having a plurality of movable leaves arranged around a central opening, the plurality of movable leaves is configured to move to adjust a size of the central opening.

In addition or alternatively to any example described herein, the electronically adjustable orifice is disposed within the handle.

In addition or alternatively to any example described herein, the electronically adjustable orifice is disposed outside of the handle.

In addition or alternatively to any example described herein, the control circuitry is in electronic communication with the electronically adjustable orifice.

In addition or alternatively to any example described herein, a surgical fluid management system may comprise an inflow pump, a fluid source line for fluidly connecting the inflow pump to a fluid source, a fluid inflow line extending downstream from the inflow pump, the fluid inflow line configured to be fluidly connected to an inflow port of a medical device, a controller configured to control the inflow pump, an electronically adjustable orifice located along the fluid inflow line, and control circuitry for receiving a signal of a current size of the electronically adjustable orifice and/or for sending a signal to change a size of the electronically adjustable orifice. The controller may be in electronic communication with the inflow pump and the electronically adjustable orifice.

In addition or alternatively to any example described herein, the surgical fluid management system may further comprise a first pressure sensor disposed upstream of the electronically adjustable orifice and a second pressure sensor disposed downstream of the electronically adjustable orifice.

In addition or alternatively to any example described herein, the control circuity is configured to calculate an approximate current flow rate of a fluid passing through the electronically adjustable orifice based on the current size of the electronically adjustable orifice, a first fluid pressure measured by the first pressure sensor, and a second fluid pressure measured by the second pressure sensor.

In addition or alternatively to any example described herein, the control circuitry is configured to calculate an approximate current flow rate of a fluid passing through the electronically adjustable orifice based on the current size of the electronically adjustable orifice and a system pressure of the fluid measured between the inflow pump and the electronically adjustable orifice.

In addition or alternatively to any example described herein, the electronically adjustable orifice includes an adjustable iris having a plurality of movable leaves arranged around a central opening, the plurality of movable leaves is configured to move to adjust a size of the central opening.

In addition or alternatively to any example described herein, an endoscopic system may comprise a fluid management system including a fluid source, an inflow pump, a fluid source line fluidly connecting the fluid source to the inflow pump, a fluid inflow line extending downstream from the inflow pump, and a controller for controlling the inflow pump, an endoscope including a handle and an elongate shaft extending distally from the handle, wherein the handle includes an inflow port in fluid communication with the elongate shaft, the inflow port being fluidly connectable to the fluid inflow line, an electronically adjustable orifice associated with the inflow port, a first pressure sensor disposed upstream of the electronically adjustable orifice, a second pressure sensor disposed downstream of the electronically adjustable orifice and upstream of the elongate shaft, and control circuitry for changing a size of the electronically adjustable orifice. The controller may be in electronic communication with the inflow pump and the electronically adjustable orifice.

In addition or alternatively to any example described herein, the control circuitry is configured to receive a signal of a current size of the electronically adjustable orifice.

In addition or alternatively to any example described herein, the control circuity is configured to calculate an approximate current flow rate of a fluid passing through the electronically adjustable orifice based on the current size of the electronically adjustable orifice, a first fluid pressure measured by the first pressure sensor, and a second fluid pressure measured by the second pressure sensor.

In addition or alternatively to any example described herein, the electronically adjustable orifice includes an adjustable iris having a plurality of movable leaves arranged around a central opening, the plurality of movable leaves is configured to move to adjust a size of the central opening.

In addition or alternatively to any example described herein, the electronically adjustable orifice is disposed within the handle.

In addition or alternatively to any example described herein, the electronically adjustable orifice is disposed outside of the handle.

In addition or alternatively to any example described herein, the control circuitry is disposed within the handle. The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic illustration of selected aspects of an endoscope;

FIG. 2 is a schematic illustration of selected aspects of an endoscopic system;

FIG. 3 is a schematic illustration of selected aspects of an endoscopic system;

FIG. 4 is a schematic illustration of selected aspects of an endoscopic system;

FIG. 5 is a schematic illustration of selected aspects of an endoscopic system;

FIG. 6 is a schematic illustration of selected aspects of an endoscopic system and/or a fluid management system;

FIG. 7 is a schematic illustration of selected aspects of an endoscopic system and/or a fluid management system;

FIG. 8 is a schematic illustration of selected aspects of a fluid management system;

FIG. 9 is a schematic illustration of selected aspects of an endoscopic system;

FIG. 10 is a schematic illustration of selected aspects of an endoscopic system;

FIGS. 11A-11B schematically illustrate selected aspects of an electronically adjustable orifice;

FIGS. 12A-12B schematically illustrate selected aspects of an electronically adjustable orifice.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, every feature and/or element may not be shown in each drawing.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Some fluid management systems for use in flexible ureteroscopy (fURS) procedures (e.g., ureteroscopy, percutaneous nephrolithotomy (PCNL), benign prostatic hyperplasia (BPH), transurethral resection of the prostate (TURP), etc.), gynecology, and other endoscopic procedures may attempt to regulate body cavity pressure when used in conjunction with an endoscope device using pressure and/or flow rate data from a fluid management system. During fURS procedures, the body cavity may be distended to make it easier to locate a target. In some procedures, blood and/or debris may be present in the body cavity, which may negatively affect image quality through the endoscopic device. Fluid flow (e.g., irrigation) through the endoscopic device may be used to flush the body cavity to improve image quality. In some procedures, the body cavity may be relatively small and irrigation fluid may flow continuously, which can raise intracavity fluid pressure and/or system pressure (e.g., fluid pressure within the fluid management system itself). Increased intracavity fluid pressure and/or system pressure may pose risks to the patient under some circumstances. Additionally, insertion of tools into a working channel or lumen of the endoscopic device may affect fluid flow (e.g., flow rate, pressure, etc.). As such, there is a need to maintain and/or adjust fluid flow (e.g., irrigation) into the body cavity to maintain good visualization while limiting and/or reducing intracavity fluid pressure and/or system pressure. In some instances, the physician may place a manual stopcock between the fluid source and/or the fluid management system and the endoscopic device to control fluid flow through the endoscopic device. However, drawbacks from this may include stopping fluid flow, causing system pressure to increase upstream of the stopcock, and/or the manual control may work against a fluid management system that is configured and/or attempting to control fluid flow. An electronically controlled orifice is proposed which may address one or more of these drawbacks and is described herein.

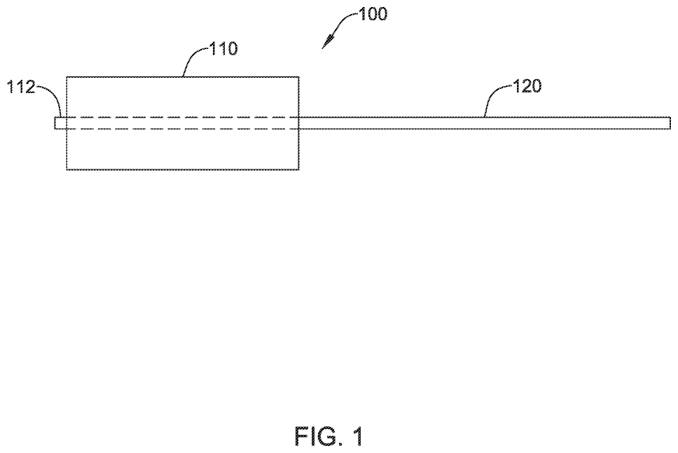

FIG. 1 is a schematic illustration of selected aspects of an endoscope 100 that may be used with an endoscopic system and/or a fluid management system of the disclosure. In some embodiments, the endoscope 100 may be one of several different types of scope device, such as a ureteroscope, a cystoscope, a nephroscope, a hysteroscope, a colonoscope, or another scope device. In some embodiments, the endoscope 100 may be a LithoVue™ scope device, or other endoscope. Other medical devices are contemplated for use with the endoscopic systems and/or the fluid management systems of the disclosure.

The endoscope 100 may include a handle 110 and an elongate shaft 120 extending distally from the handle 110. The handle 110 of the endoscope 100 may include an inflow port 112 in fluid communication with the elongate shaft 120 and/or one or more working lumens extending within the elongate shaft 120 to deliver fluid through the elongate shaft 120 to a distal end of the elongate shaft 120. The inflow port 112 may be sized and configured to fluidly connect to a fluid inflow line, as will be discussed herein. In some embodiments, the inflow port 112 may include a Luer connector, a threaded connector, a snap connector, or another suitable connector type.

In some embodiments, specific features and/or configurations of the endoscope 100 may vary. In some embodiments, the handle 110 may have a fluid flow on/off switch, which may allow the user to control when fluid is flowing through the endoscope 100 and into the treatment site from a fluid source and/or a fluid management system. The handle 110 may further include other buttons that perform other various functions. For example, in some embodiments, the handle 110 may include buttons to control the temperature of fluid provided by a fluid management system and/or deflection of a distal tip of the elongate shaft 120. In some embodiments, a medical instrument or tool used during a procedure may be inserted into the one or more working lumens of the endoscope 100 through a working lumen access port.

In some embodiments, the endoscope 100 may be configured to deliver fluid to a treatment site via the elongate shaft 120. The elongate shaft 120 may be configured to access the treatment site within the patient. In some embodiments, a fluid source may be in fluid communication with the endoscope 100 and/or the elongate shaft 120, as will be discussed herein. The elongate shaft 120 may include the one or more working lumens for receiving a flow of fluid and/or other medical devices therethrough. The endoscope 100 may be connected to a fluid source and/or a fluid management system via one or more supply line(s) (e.g., the fluid inflow line).

In some embodiments, the endoscope 100 may be in electronic communication with a workstation via a wired connection or a wireless connection. The workstation may include a touch panel computer, an interface box for receiving the wired connection and/or wireless communications, a cart, and a power supply, among other features. In some embodiments, the interface box may be configured with a wired or wireless communication connection with a controller of a fluid management system. The touch panel computer may include at least a display screen and an image processor. In some embodiments, the workstation may be a multi-use component (e.g., used for more than one procedure) while the endoscope 100 may be a single use device, although this is not required. In some embodiments, the workstation may be omitted and the endoscope 100 may be electronically coupled directly to a controller of a fluid management system.

In some embodiments, the one or more supply line(s) from the fluid management system to the endoscope 100 may be formed of a material the helps dampen the peristaltic motion created by an inflow pump of the fluid management system. In some embodiments, the supply line(s) may be formed from small diameter tubing less than or equal to 1/16 inches (1.5875 millimeters) in diameter. However, it will be understood that tubing size may vary based on the application. The supply line(s) and/or the tubing may be disposable and provided sterile and ready to use. Different types of tubing may be used for various functions within the fluid management system. For example, one type of tubing may be used for fluid heating and fluid flow control to the endoscope 100 while another type of tubing may be used for irrigation within the body and/or the treatment site.

In some embodiments, the endoscope 100 may include one or more sensors proximate a distal end of the elongate shaft 120. For example, the endoscope 100 may include a distal pressure sensor at a distal end of the elongate shaft 120 to measure intracavity pressure within the treatment site. The endoscope 100 may also include other sensors such as, for example, a distal temperature sensor, a Fiber Bragg grating optical fiber to detect stresses, and/or an antenna or electromagnetic sensor (e.g., a position sensor). In some embodiments, the distal end of the elongate shaft 120 of the endoscope 100 may also include at least one camera to provide a visual feed to the user on the display screen of the touch panel computer. In another embodiment, the endoscope 100 may include two cameras having different communications requirements or protocols so that different information may be relayed to the user by each camera. When so provided, the user may switch back and forth between cameras at will through the touch screen interface and/or the touch panel computer.

In some embodiments, the location of the distal end of the elongate shaft 120 may be tracked during use. For example, a mapping and navigation system may include an operating table (or other procedural or examination table or chair, etc.) configured to act or function as an electromagnetic generator to generate a magnetic field of a known geometry. Alternatively, or additionally, an electromagnetic generator separate from the operating table may be provided. The operating table and/or the electromagnetic generator may be coupled to a control unit which may include among other features, a processor, a memory, a display, and an input means. A position sensor (e.g., the electromagnetic sensor, etc.) or other antenna, may be incorporated into the distal end of the elongate shaft 120 of the endoscope 100. The position sensor may be configured for use in sensing a location of the position sensor in the magnetic field of the mapping and navigation system. In some embodiments, the position sensor may be electronically coupled to the workstation. When the position sensor is in the magnetic field, the location of the position sensor can be mathematically determined relative to the electromagnetic field source (e.g., the operating table and/or the electromagnetic generator). The workstation and the control unit may communicate to determine the position of the position sensor relative to the patient.

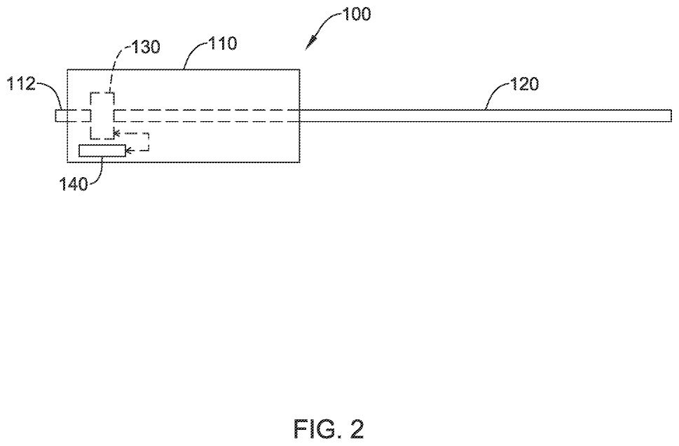

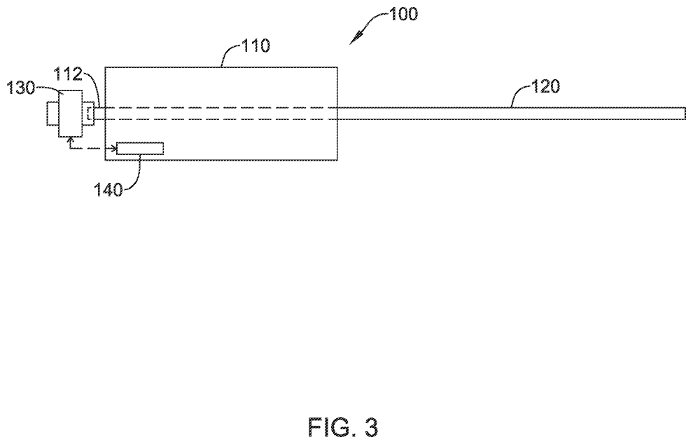

FIGS. 2-5 schematically illustrate selected aspects and/or configurations of an endoscopic system. The endoscopic system may include the endoscope 100, as described herein. In some embodiments, the endoscopic system may include an electronically adjustable orifice 130 associated with the inflow port 112 of the handle 110. In some embodiments, the electronically adjustable orifice 130 may be disposed within the handle 110, as shown in FIG. 2. In some embodiments, the electronically adjustable orifice 130 may be disposed outside of the handle 110, as shown in FIG. 3. In some embodiments, the electronically adjustable orifice 130 may be integrated into the handle 110. In some embodiments, the electronically adjustable orifice 130 may be a separate component that is attachable to the inflow port 112 of the handle 110. In some embodiments, the electronically adjustable orifice 130 may include one or more motors, gears, cams, pulleys, etc. configured to and/or capable of managing, maintaining, and/or changing a size of the electronically adjustable orifice 130.

The endoscopic system may include controls 140 associated with the handle 110. In some embodiments, the controls 140 may be integrally formed in the handle 110. In some embodiments, the controls 140 may be fixedly secured to an outer surface of the handle 110. In some embodiments, the controls 140 may be a separate element that may be added on to the endoscope 100 and/or the handle 110. In some embodiments, the controls 140 may be removably secured to the outer surface of the handle 110. Other configurations are also contemplated. The endoscopic system may include control circuitry for receiving a signal of a current size of the electronically adjustable orifice 130 and/or for sending a signal to change a size of the electronically adjustable orifice 130. In at least some embodiments, the control circuitry may be associated with the controls 140. In some embodiments, the control circuitry may be disposed within the handle 110 of the endoscope 100. In some embodiments, the control circuitry may be disposed outside of the handle 110 of the endoscope 100. In some embodiments, the control circuitry may be in electronic communication with the controls 140. In some embodiments, the controls 140 and/or the control circuitry may be in electronic communication with the electronically adjustable orifice 130, as indicated by a dashed line between these elements in the figures. In some embodiments, the controls 140 and/or the control circuitry may be hardwired to the electronically adjustable orifice 130. In some embodiments, the controls 140 and/or the control circuitry may be wirelessly connected to and/or in wireless communication with the electronically adjustable orifice 130. Other configurations are also contemplated.

Accordingly, a user of the endoscope 100 may be able to use the controls 140 to change the size of the electronically adjustable orifice 130. For example, when the user actuates the controls 140, the control circuitry may send a signal to the electronically adjustable orifice 130 to change the size of the electronically adjustable orifice 130. In some embodiments, the controls 140 may include one or more of speed control, home position, fully open, fully closed, increase size, decrease size, etc. In some embodiments, the controls 140 may include buttons, dials, knobs, slides, touch interface(s), voice interface(s), etc. In some embodiments, the controls 140 may include multiple different types of controls (e.g., buttons and knobs, etc.).

In some embodiments, the endoscopic system may include a first pressure sensor 150 disposed upstream of the electronically adjustable orifice 130. In some embodiments, the first pressure sensor 150 may be disposed immediately upstream of the electronically adjustable orifice 130. In some embodiments, the endoscopic system may include a second pressure sensor 160 disposed downstream of the electronically adjustable orifice 130 and upstream of the elongate shaft 120 of the endoscope 100. In some embodiments, the second pressure sensor 160 may be disposed immediately downstream of the electronically adjustable orifice 130 and upstream of the elongate shaft 120 of the endoscope 100.

In some embodiments, the first pressure sensor 150 and/or the second pressure sensor 160 may be integrated into the handle 110 of the endoscope 100. In some embodiments, the first pressure sensor 150 and/or the second pressure sensor 160 may be integrated into the electronically adjustable orifice 130 to form an electronically adjustable orifice assembly. In some embodiments, the first pressure sensor 150 and/or the second pressure sensor 160 may be standalone elements added to and/or connected to the endoscope 100 and/or the electronically adjustable orifice 130. In some embodiments, the first pressure sensor 150 and/or the second pressure sensor 160 may be releasably connected to the endoscope 100 and/or the electronically adjustable orifice 130.

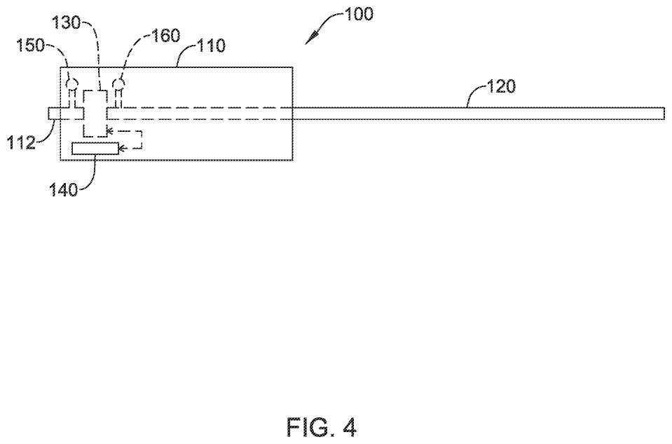

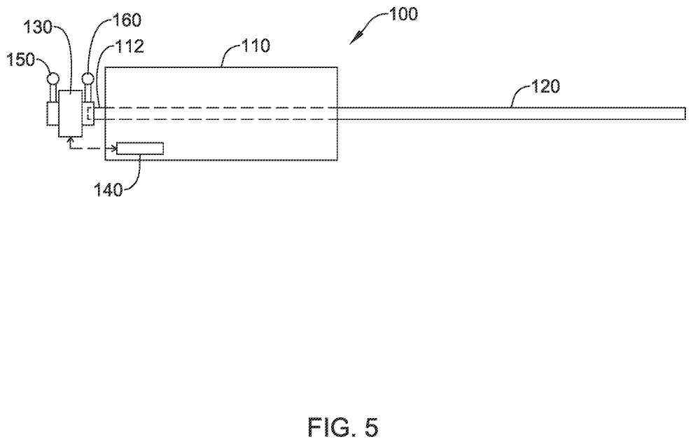

In some embodiments, the first pressure sensor 150 and/or the second pressure sensor 160 may be disposed downstream of the inflow port 112, as seen in FIG. 4. In some embodiments, the electronically adjustable orifice assembly may be disposed downstream of the inflow port 112. In some embodiments, the first pressure sensor 150 and/or the second pressure sensor 160 may be disposed upstream of the inflow port 112, as seen in FIG. 5. Other configurations are also contemplated.

The first pressure sensor 150 may be configured to measure a first fluid pressure of fluid flowing through the endoscope 100 upstream of the electronically adjustable orifice 130. Preferably, the first pressure sensor 150 may be configured to measure a first fluid pressure of fluid flowing into the electronically adjustable orifice 130 immediately before the fluid enters the electronically adjustable orifice 130. The second pressure sensor 160 may be configured to measure a second fluid pressure of fluid flowing through the endoscope 100 downstream of the electronically adjustable orifice 130. Preferably, the second pressure sensor 160 may be configured to measure a second fluid pressure of fluid flowing out of the electronically adjustable orifice 130 immediately after the fluid exits or leaves the electronically adjustable orifice 130.

Location of the first pressure sensor 150 and the second pressure sensor 160 relative to the electronically adjustable orifice 130 may be pertinent to operation of the endoscopic system and/or the electronically adjustable orifice 130. For example, by locating the first pressure sensor 150 and the second pressure sensor 160 physically close to the electronically adjustable orifice 130 (e.g., within about 5-50 millimeters), the control circuitry may be configured to calculate and/or estimate an approximate current flow rate of the fluid passing through the electronically adjustable orifice 130 based on the current size of the electronically adjustable orifice 130, the first fluid pressure measured by the first pressure sensor 150, and the second fluid pressure measured by the second pressure sensor 160. Fluid flow rate may be calculated and/or estimated based on a combination of known and measured characteristics, as is known in the art. In some embodiments, the first pressure sensor 150 and/or the second pressure sensor 160 may send pressure signals to the control circuitry to facilitate and/or trigger changes in state or system behavior with instantaneous or near-instantaneous response.

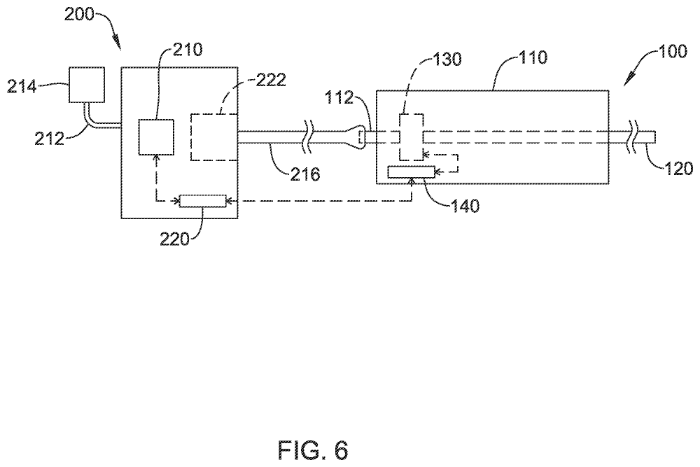

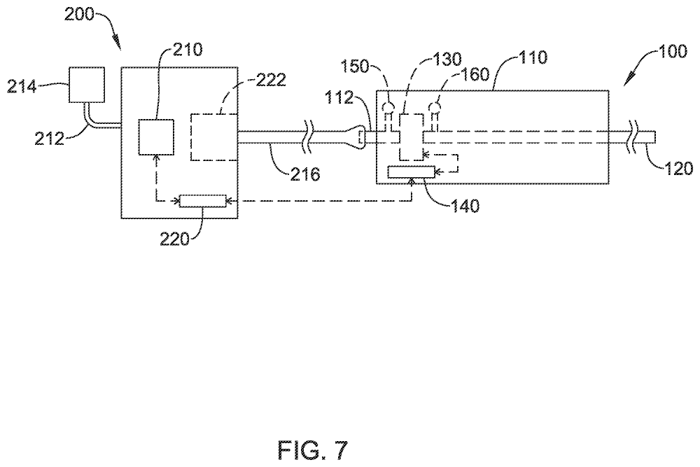

FIGS. 6-7 schematically illustrate selected aspects and/or configurations of an endoscopic system. The endoscopic system may include an endoscope 100, as described herein. In some embodiments, the endoscopic system may include the electronically adjustable orifice 130 associated with the inflow port 112 of the handle 110. In some embodiments, the electronically adjustable orifice 130 may be disposed within the handle 110, as shown in FIGS. 2 and 6. In some embodiments, the electronically adjustable orifice 130 may alternatively be disposed outside of the handle 110, as shown in FIG. 3. Other configurations are also contemplated.

Returning to FIG. 6, the endoscopic system may include a surgical fluid management system 200. The fluid management system 200 may include a fluid source 214 (e.g., a saline bag or other fluid bag), an inflow pump 210, a fluid source line 212 fluidly connecting the fluid source 214 to the inflow pump 210, a fluid inflow line 216 extending downstream from the inflow pump 210 and/or the fluid management system 200, and a controller 220 configured to control the inflow pump 210 and/or the fluid management system 200. In some embodiments, the fluid inflow line 216 may be configured to be fluidly connected to an inflow port (e.g., the inflow port 112) of a medical device (e.g., the endoscope 100, etc.).

In some embodiments, the inflow pump 210 may be configured to pump and/or transfer fluid from the fluid source 214 (e.g., a fluid bag, a reservoir, etc.) to the endoscope 100 and/or a treatment site within a patient at a fluid flow rate. In some embodiments, the fluid management system 200 may optionally include a fluid warming system 222, described in more detail below.

The flow of fluid, the system pressure of the fluid, the temperature of the fluid, and/or other operational parameters may be controlled by or at least partially controlled by the controller 220. The controller 220 may be in electronic communication (e.g., wired or wireless) with the endoscope 100, the electronically adjustable orifice 130, the control circuitry, the inflow pump 210, and/or the fluid warming system 222 to provide control commands and/or to transfer or receive data therebetween. For example, the controller 220 may receive data such as, but not limited to, pressure signals, temperature data, orifice size, etc. In some embodiments, the controller 220 and/or the control circuitry may be configured to receive a signal of a current size of the electronically adjustable orifice 130. As such, in at least some embodiments, the controller 220 and/or the fluid management system 200 may “know” the current size of the electronically adjustable orifice 130 at any time during a procedure. In some embodiments, the controller 220 and/or the control circuitry may be configured to send a signal to change a size of the electronically adjustable orifice 130. In some embodiments, the controller 220 and/or the control circuitry may be configured to send a signal to change a size of the electronically adjustable orifice 130 when instructed by the controls 140 and/or the user. In some embodiments, the controller 220 and/or the control circuitry may be configured to send a signal to change a size of the electronically adjustable orifice 130 automatically based on preset and/or operational parameters of the fluid management system 200. Other configurations are also contemplated. In some embodiments, the controller 220 may use the received data to control operational parameters of the inflow pump 210 and/or the fluid warming system 222. In some embodiments, the controller 220 may send signals and/or instructions to the control circuitry.

In some embodiments, the fluid management system 200 may include one or more user interface components such as one or more knobs, one or more switches, and/or a touch screen interface. The touch screen interface may include a display and may include switches or knobs in addition to touch capabilities. In some embodiments, the controller 220 may include the touch screen interface and/or the display. The touch screen interface may allow the user to input/adjust various functions of the fluid management system 200 such as, for example system fluid pressure, fluid temperature, or inflow pump speed (e.g., rpm) which may correlate to flow rate. The user may also configure parameters and alarms (such as, but not limited to, a system pressure limit, an inflow pump speed limit, etc.), information to be displayed, etc. The touch screen interface may allow the user to add, change, and/or discontinue the use of various modular systems within the fluid management system 200. The touch screen interface may also be used to change the fluid management system 200 between automatic and manual modes for various procedures. It is contemplated that other systems configured to receive user input may be used in place of or in addition to the touch screen interface.

In some embodiments, the touch screen interface may be configured to include selectable areas like buttons and/or may provide a functionality similar to physical buttons as would be understood by those skilled in the art. The display may be configured to show icons related to modular systems and devices included in the fluid management system 200. In some embodiments, the display may include an estimated flow rate display. The estimated flow rate display may be determined based on based on the current size of the electronically adjustable orifice 130, the first fluid pressure measured by the first pressure sensor 150, and the second fluid pressure measured by the second pressure sensor 160, and/or other known values or characteristics.

In some embodiments, the operating parameters may be adjusted by touching a corresponding portion of the touch screen interface. The touch screen interface and/or the display may also display visual alerts and/or issue audio alarms if parameters (e.g., pump speed, system pressure, fluid temperature, etc.) are above or below predetermined thresholds and/or ranges. The touch screen interface and/or the display may also be configured to display any other information the user may find useful during the procedure. In some embodiments, the fluid management system 200 may also include further user interface components such as a heater user interface, a fluid control interface, or other device to manually control various modular systems.

The touch screen interface may be operatively connected to or may be a part of the controller 220. The controller 220 may be a computer, tablet computer, or other processing device. The controller 220 may be operatively connected to one or more system components such as, for example, the inflow pump 210, the fluid warming system 222, a fluid deficit management system, etc. In some embodiments, these features may be integrated into a single unit. The controller 220 is capable of and configured to perform various functions such as calculation, control, computation, display, etc. The controller 220 is also capable of tracking and storing data pertaining to the operations of the fluid management system 200 and each component thereof. In an illustrative embodiment, the controller 220 includes wired and/or wireless network communication capabilities, such as ethernet or Wi-Fi, through which the controller 220 may be connected to, for example, a local area network. The controller 220 may also receive signals from one or more of the sensors of the fluid management system 200, the endoscope 100, the electronically adjustable orifice 130, and/or the electronically adjustable orifice assembly. In some embodiments, the controller 220 may communicate with databases for best practice suggestions and the maintenance of patient records which may be displayed to the user on the display.

In some embodiments, the inflow pump 210 may be a peristaltic pump. In some embodiments, the inflow pump 210 may include multiple pumps or more than one pump. The inflow pump 210 may be electrically driven and may receive power from a line source such as a wall outlet, an external or internal electrical storage device such as a disposable or rechargeable battery, and/or an internal power supply. The inflow pump 210 may operate at any desired speed sufficient to deliver fluid at a target system pressure and/or at an estimated fluid flow rate. In some embodiments, the controller 220 may be configured to automatically adjust one or more outputs for controlling the inflow pump 210.

In some embodiments, the one or more outputs for controlling the inflow pump 210 may also be manually adjusted via, for example, the touch screen interface or a separate fluid controller. While not explicitly shown, the controller 220 may include a separate user interface including buttons that allow the user to increase or decrease the speed and/or the output of the inflow pump 210. In some embodiments, the fluid management system 200 may include multiple pumps having different flow capabilities. Since parameters and/or characteristics of the fluid management system 200 are generally known in advance, inflow pump speed may be correlated to flow rate within the fluid management system 200. In addition or alternatively, in some embodiments, the fluid management system 200 may optionally include a flow rate sensor to measure actual fluid flow rate. The flow rate sensor may be operably connected to the controller 220 and data from the flow rate sensor may be used by the controller 220 to change selected system parameters.

Inflow pump speed, estimated fluid flow rate, actual fluid flow rate, and/or system pressure at any given time may be displayed on the display to allow the operating room (OR) visibility for any changes. If the OR personnel notice a change in inflow pump speed, estimated fluid flow rate, actual fluid flow rate, and/or system pressure that is either too high or too low, the user may manually adjust one or more outputs for controlling the inflow pump 210 and/or the inflow pump speed, estimated fluid flow rate, actual fluid flow rate, and/or system pressure, back to a preferred level. In some embodiments, the fluid management system 200 and/or the controller 220 may monitor and automatically adjust one or more outputs for controlling the inflow pump 210.

In some embodiments, the fluid management system 200 may optionally include the fluid warming system 222 for heating fluid to be delivered to the patient and/or the treatment site. The fluid warming system 222 may include a heater and a heater cassette. The heater cassette may be configured to be a single use heater cassette while the heater may be reused for multiple procedures. For example, the heater cassette may isolate fluid flow such that the heater may be reused with minimal maintenance. The heater cassette may be formed of, for example, polycarbonate or any high heat rated biocompatible plastic and is formed as a single unitary and/monolithic piece or a plurality of pieces permanently bonded to one another. In some embodiments, the heater cassette may include a fluid inlet port and a fluid outlet port located at a lateral side of the heater cassette. The fluid inlet port and the fluid outlet port may each be configured to couple to the supply line(s) of the fluid management system 200. For example, the fluid inlet port may couple the heater cassette and/or the fluid warming system 222 to the fluid source 214 (via the inflow pump 210) using the supply line(s) and/or the fluid source line 212, while the fluid outlet port may couple the fluid warming system 222 with the endoscope 100 via the fluid inflow line 216.

In some embodiments, the heater cassette may include an internal flow path along a channel through which fluid may flow from the fluid inlet port to the fluid outlet port. The heater cassette, the channel, and/or the internal flow path may include one fluid flow path or multiple fluid flow paths. In some embodiments, the channel may pass through a susceptor which may allow the fluid to be heated via induction heating. When the heater cassette is coupled with the heater, the susceptor may be configured to be positioned within an induction coil. Other fluid warming system configurations and methods may also be used, as desired. For example, the heater may include one or more heat sources such as, for example a platen system or an inline coil in the supply line(s) using electrical energy. Heating may be specifically designed and tailored to the inflow pump speed, fluid flow rates, and/or system pressure required in the specific application of the fluid management system 200. Some illustrative fluid warming systems are described in described in commonly assigned U.S. MANAGEMENT SYSTEM, the entire disclosure of which is hereby incorporated by reference.

In some embodiments, the fluid warming system 222 may include a heater user interface separate from the touch screen interface. The heater user interface may simply be a display screen providing a digital display of the internal temperature of the heater. In another embodiment, the user interface, the controls 140, and/or the endoscope 100 may also include temperature adjustment buttons to increase or decrease the temperature of the heater. In some embodiments, the heater user interface and/or the display screen may indicate the current temperature of the heater as well as the target temperature to be reached. It is noted that all information output from the fluid warming system 222 may be transmitted directly to the display such that no heater user interface is necessary.

The fluid warming system 222 may include one or more sensors configured to monitor the fluid flowing therethrough. For example, temperature sensors may be mounted in the fluid warming system 222 such that they detect the temperature of the fluid flowing through the heater cassette. The temperature sensors may be located at or near the fluid inlet port and/or the fluid outlet port. In some embodiments, the temperature sensors may be mounted so that they detect the temperature of fluid flowing through the heater cassette prior to the fluid entering the susceptor and after fluid exits the susceptor. In some embodiments, additional sensors may be located at a medial portion of the susceptor so that they detect a progression of temperature increase of the fluid in the heater cassette. The temperature sensors may remotely send any information to the display or they may send information to heater user interface and/or the display screen thereof, if so provided. In another embodiment, the temperature sensors may be hardwired with the heater user interface (if provided) which is then able to remotely transmit desired information to the display. Alternatively, or additionally, the temperature sensors may be hardwired to and/or with the controller 220.

The heater may further include at least one pressure sensor configured to monitor system pressure and/or a bubble sensor configured to monitor the fluid flowing through the system for bubbles. The heater cassette may include a corresponding pressure sensor interface and bubble sensor interface that allow the at least one pressure sensor and the bubble sensor, respectively, to monitor the fluid flowing through the heater cassette when the heater cassette is coupled with the fluid warming system 222. The at least one pressure sensor and/or the bubble sensor may remotely and/or electronically send data and/or information to the controller 220, to the display, and/or to the heater user interface and/or the display screen thereof, if so provided. The controller 220 may be configured to receive pressure signals from the at least one pressure sensor, the pressure signals corresponding to a system pressure within the fluid management system 200. In some embodiments, the at least one pressure sensor and/or the bubble sensor may be hardwired with the heater user interface (if provided) which is then able to remotely transmit desired information to the display. Alternatively, or additionally, the at least one pressure sensor and/or the bubble sensor may be hardwired to and/or with the controller 220.

In some embodiments, the at least one pressure sensor may include one pressure sensor, two pressure sensors, three pressure sensors, or more pressure sensors. In some embodiments having two or more pressure sensors, the individual pressure sensors may be spaced apart from each other. In some embodiments, the at least one pressure sensor may be positioned downstream of the inflow pump 210. In some embodiments, the at least one pressure sensor may be positioned upstream of the fluid inflow line 216. In some embodiments, the at least one pressure sensor may be positioned downstream of the inflow pump 210 and upstream of the fluid inflow line 216. In some embodiments, the at least one pressure sensor may be configured to detect the system pressure within the fluid management system 200 downstream of the inflow pump 210 and/or upstream of the fluid inflow line 216.

In some embodiments, the heater cassette may collectively act as a fluid reservoir. In some embodiments, the fluid reservoir of the heater cassette may include a pulsation dampener to reduce peristaltic pulsations, and one or more air traps to remove bubbles before and/or after heating the fluid flowing through the heater cassette. In some embodiments, the pulsation dampener and the one or more air traps may collectively act as the fluid reservoir. Fluid level(s) within the fluid reservoir of the heater cassette may rise and fall based on a ratio between an inflow amount of fluid being pumped into the heater cassette and an outflow amount of fluid exiting the heater cassette (e.g., flowing to the endoscope 100 and/or the patient).

In each configuration, the fluid management system 200 may operate in one or more different modes—for example, a “pressure control mode”, a “flow compensation mode”, etc.

In the pressure control mode, the controller 220 may modulate various system parameters and/or the one or more outputs to the inflow pump 210 to keep and/or maintain the system pressure at a system pressure set point, which may be entered by the user on the touch screen interface. In some embodiments, the system pressure set point may be set and/or selected automatically based on which type and/or configuration of endoscope 100 is fluidly connected to the inflow pump 210. As discussed herein, the system pressure may be measured by the at least one pressure sensor within the fluid management system 200.

In some embodiments, the fluid management system 200 may be fluidly connected to a working lumen of the endoscope 100. As such, the fluid management system 200 may be configured to control an inflow of fluid from the fluid management system 200 through the endoscope 100 to the treatment site. In at least some embodiments, the working lumen of the endoscope 100 may also be used to insert a medical instrument or tool through the endoscope 100 to the treatment site. Insertion of the medical instrument or tool may partially obstruct the working lumen and thus affect the flow and/or pressure characteristics of the inflow of fluid. In the flow compensation mode, the fluid management system 200 and/or the controller 220 may be configured to automatically modulate selected system parameters to attempt to maintain a desired or estimated flow rate through the fluid inflow line 216 and/or the endoscope 100. Thus, the fluid management system 200 may be configured to attempt to overcome the partial obstruction of the working lumen. As such, communication with the electronically adjustable orifice 130 and/or the current size of the electronically adjustable orifice 130 may be relevant to operation of the fluid management system 200. Accordingly, the fluid management system 200 and/or the controller 220 may be in electronic communication with the inflow pump 210, the controls 140, the control circuitry, and/or the electronically adjustable orifice 130, as indicated by dashed lines in the figures.

In some embodiments, the control circuitry and/or the controller 220 may be configured to receive a signal of a current size of the electronically adjustable orifice 130 and/or may be configured to send a signal to change a size of the electronically adjustable orifice 130. In some embodiments, the control circuitry and/or the controller 220 may be configured to calculate and/or estimate an approximate current flow rate of a fluid passing through the electronically adjustable orifice 130 based on the current size of the electronically adjustable orifice 130 and a system pressure of the fluid measured between the inflow pump 210 and the electronically adjustable orifice 130. In some embodiments, the control circuitry and/or the controller 220 may be configured to calculate and/or estimate an approximate current flow rate of a fluid passing through the electronically adjustable orifice 130 based on the current size of the electronically adjustable orifice 130 and a system pressure of the fluid measured between the inflow pump 210 and the fluid inflow line 216.

Turning to FIG. 7, in some embodiments, the endoscopic system may include the first pressure sensor 150 disposed upstream of the electronically adjustable orifice 130. In some embodiments, the first pressure sensor 150 may be disposed immediately upstream of the electronically adjustable orifice 130. In some embodiments, the endoscopic system may include the second pressure sensor 160 disposed downstream of the electronically adjustable orifice 130 and upstream of the elongate shaft 120 of the endoscope 100. In some embodiments, the second pressure sensor 160 may be disposed immediately downstream of the electronically adjustable orifice 130 and upstream of the elongate shaft 120 of the endoscope 100.

In some embodiments, the first pressure sensor 150 and/or the second pressure sensor 160 may be integrated into the handle 110 of the endoscope 100. In some embodiments, the first pressure sensor 150 and/or the second pressure sensor 160 may be integrated into the electronically adjustable orifice 130 to form an electronically adjustable orifice assembly. In some embodiments, the first pressure sensor 150 and/or the second pressure sensor 160 may be standalone elements added to and/or connected to the endoscope 100 and/or the electronically adjustable orifice 130. In some embodiments, the first pressure sensor 150 and/or the second pressure sensor 160 may be releasably connected to the endoscope 100 and/or the electronically adjustable orifice 130.

In some embodiments, the first pressure sensor 150 and/or the second pressure sensor 160 may be disposed downstream of the inflow port 112, as seen in FIGS. 4 and 7. In some embodiments, the electronically adjustable orifice assembly may be disposed downstream of the inflow port 112. In some embodiments, the first pressure sensor 150 and/or the second pressure sensor 160 may alternatively be disposed upstream of the inflow port 112, as seen in FIG. 5. Other configurations are also contemplated.

Returning to FIG. 7, the endoscopic system may include the fluid management system 200. In at least some embodiments, the fluid management system 200 may be configured as described herein.

In some embodiments, the control circuitry and/or the controller 220 may be configured to receive a signal of a current size of the electronically adjustable orifice 130. In some embodiments, the control circuitry and/or the controller 220 may be configured to calculate and/or estimate an approximate current flow rate of a fluid passing through the electronically adjustable orifice 130 based on the current size of the electronically adjustable orifice 130, the first fluid pressure measured by the first pressure sensor 150 (upstream of the electronically adjustable orifice 130), and the second fluid pressure measured by the second pressure sensor 160 (downstream of the electronically adjustable orifice 130). In some embodiments, the control circuitry and/or the controller 220 may be configured to send a signal to change the current size of the electronically adjustable orifice 130, which may in turn change the flow rate of fluid flowing through the fluid channel of the endoscope 100.

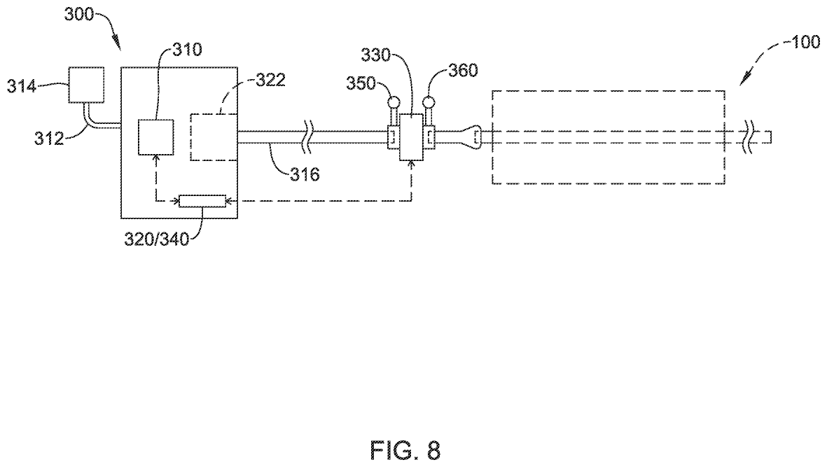

FIG. 8 schematically illustrates an example standalone surgical fluid management system 300 that may be suitable for use with a variety of different endoscopic systems and/or an endoscope 100, shown in phantom. The fluid management system 300 may include an inflow pump 310. The fluid management system 300 may include a fluid source line 312 for fluidly connecting the inflow pump 310 to a fluid source 314 (e.g., a saline bag or other fluid bag). The fluid management system 300 may include a fluid inflow line 316 extending downstream from the inflow pump 310. The fluid inflow line 316 may be configured to be fluidly connected to an inflow port (e.g., the inflow port 112) of a medical device (e.g., the endoscope 100, an endoscopic system, etc.). The fluid management system 300 may include a controller 320 configured to control the inflow pump 310.

The fluid management system 300 may include an electronically adjustable orifice 330 located along the fluid inflow line 316. In some embodiments, the electronically adjustable orifice 330 may include one or more motors, gears, cams, pulleys, etc. configured to and/or capable of managing, maintaining, and/or changing a size of the electronically adjustable orifice 330.

The fluid management system 300 may include controls 340. The fluid management system 300 may include control circuitry for receiving a signal of a current size of the electronically adjustable orifice 330 and/or for sending a signal to change a size of the electronically adjustable orifice 330. In at least some embodiments, the control circuitry may be associated with the controls 340. In some embodiments, the controls 340 and/or the control circuitry may be associated with the controller 320. In some embodiments, the control circuitry may be in electronic communication with the controls 340 and/or the controller 320. In some embodiments, the controller 320, the controls 340, and/or the control circuitry may be in electronic communication with the electronically adjustable orifice 330 and/or the inflow pump 310, as indicated by a dashed line between these elements in the figures. In some embodiments, the controller 320, the controls 340, and/or the control circuitry may be hardwired to the electronically adjustable orifice 330. In some embodiments, the controller 320, the controls 340, and/or the control circuitry may be wirelessly connected to and/or in wireless communication with the electronically adjustable orifice 330. Other configurations are also contemplated.

Accordingly, a user of the fluid management system 300 may be able to use the controls 340 to change the size of the electronically adjustable orifice 330, which in turn may change the flow rate of the fluid through the orifice 330 and through the fluid channel of the endoscope 100. For example, when the user actuates the controls 340, the control circuitry may send a signal to the electronically adjustable orifice 330 to change the size of the electronically adjustable orifice 330. In some embodiments, the controls 340 may include one or more of speed control, home position, fully open, fully closed, increase size, decrease size, etc. In some embodiments, the controls 340 may include buttons, dials, knobs, slides, touch interface(s), voice interface(s), etc. In some embodiments, the controls 340 may include multiple different types of controls (e.g., buttons and knobs, etc.).

In some embodiments, the fluid management system 300 may include a first pressure sensor 350 disposed upstream of the electronically adjustable orifice 330. In some embodiments, the first pressure sensor 350 may be disposed immediately upstream of the electronically adjustable orifice 330. In some embodiments, the endoscopic system may include a second pressure sensor 360 disposed downstream of the electronically adjustable orifice 330 and upstream of a connected medical device (e.g., the endoscope 100, etc.). In some embodiments, the second pressure sensor 360 may be disposed immediately downstream of the electronically adjustable orifice 330 and upstream of the connected medical device (e.g., the endoscope 100, etc.).

In some embodiments, the first pressure sensor 350 and/or the second pressure sensor 360 may be integrated into the electronically adjustable orifice 330 to form an electronically adjustable orifice assembly. In some embodiments, the first pressure sensor 350 and/or the second pressure sensor 360 may be standalone elements added to and/or connected to the fluid management system 300 and/or the electronically adjustable orifice 330. In some embodiments, the first pressure sensor 350 and/or the second pressure sensor 360 may be releasably connected to the fluid management system 300 and/or the electronically adjustable orifice 330.

In some embodiments, the first pressure sensor 350 and/or the second pressure sensor 360 may be disposed upstream of an inflow port (e.g., the inflow port 112) of the connected medical device (e.g., the endoscope 100, etc.). In some embodiments, the electronically adjustable orifice assembly may be disposed upstream of an inflow port (e.g., the inflow port 112) of the connected medical device (e.g., the endoscope 100, etc.).

The first pressure sensor 350 may be configured to measure a first fluid pressure of fluid flowing through the fluid management system 300 upstream of the electronically adjustable orifice 330 and downstream of the inflow pump 310. Preferably, the first pressure sensor 350 may be configured to measure a first fluid pressure of fluid flowing into the electronically adjustable orifice 330 immediately before the fluid enters the electronically adjustable orifice 330. The second pressure sensor 360 may be configured to measure a second fluid pressure of fluid flowing through the fluid management system 300 downstream of the electronically adjustable orifice 330. Preferably, the second pressure sensor 360 may be configured to measure a second fluid pressure of fluid flowing out of the electronically adjustable orifice 330 immediately after the fluid exits or leaves the electronically adjustable orifice 330.

Location of the first pressure sensor 350 and the second pressure sensor 360 relative to the electronically adjustable orifice 330 may be pertinent to operation of the fluid management system 300 and/or the electronically adjustable orifice 330. For example, by locating the first pressure sensor 350 and the second pressure sensor 360 physically close to the electronically adjustable orifice 330 (e.g., within about 5-50 millimeters), the control circuitry may be configured to calculate and/or estimate an approximate current flow rate of the fluid passing through the electronically adjustable orifice 330 based on the current size of the electronically adjustable orifice 330, the first fluid pressure measured by the first pressure sensor 350, and the second fluid pressure measured by the second pressure sensor 360. Fluid flow rate may be calculated and/or estimated based on a combination of known and measured characteristics, as is known in the art. In some embodiments, the first pressure sensor 350 and/or the second pressure sensor 360 may send pressure signals to the control circuitry to facilitate and/or trigger changes in state or system behavior with instantaneous or near-instantaneous response.

In some embodiments, the inflow pump 310 may be configured to pump and/or transfer fluid from the fluid source 314 (e.g., a fluid bag, a reservoir, etc.) to a connected medical device (e.g., an endoscope 100, etc.) at a fluid flow rate. In some embodiments, the fluid management system 300 may optionally include a fluid warming system 322, described in more detail below.

The flow of fluid, the system pressure of the fluid, the temperature of the fluid, and/or other operational parameters may be controlled by or at least partially controlled by the controller 320. The controller 320 may be in electronic communication (e.g., wired or wireless) with a connected medical device (e.g., an endoscope 100, etc.), the electronically adjustable orifice 330, the controls 340, the control circuitry, the inflow pump 310, and/or the fluid warming system 322 to provide control commands and/or to transfer or receive data therebetween. For example, the controller 320 may receive data such as, but not limited to, pressure signals, temperature data, orifice size, etc. In some embodiments, the controller 320 and/or the control circuitry may be configured to receive a signal of a current size of the electronically adjustable orifice 330. As such, in at least some embodiments, the controller 320 and/or the fluid management system 300 may “know” the current size of the electronically adjustable orifice 330 at any time during a procedure. In some embodiments, the controller 320 and/or the control circuitry may be configured to send a signal to change a size of the electronically adjustable orifice 330. In some embodiments, the controller 320 and/or the control circuitry may be configured to send a signal to change a size of the electronically adjustable orifice 330 when instructed by the controls 340 and/or the user. In some embodiments, the controller 320 and/or the control circuitry may be configured to send a signal to change a size of the electronically adjustable orifice 330 automatically based on preset and/or operational parameters of the fluid management system 300. Other configurations are also contemplated. In some embodiments, the controller 320 may use the received data to control operational parameters of the inflow pump 310 and/or the fluid warming system 322. In some embodiments, the controller 320 may send signals and/or instructions to the control circuitry.

In some embodiments, the fluid management system 300 may include one or more user interface components such as one or more knobs, one or more switches, and/or a touch screen interface. The touch screen interface may include a display and may include switches or knobs in addition to touch capabilities. In some embodiments, the controller 320 may include the touch screen interface and/or the display. The touch screen interface may allow the user to input/adjust various functions of the fluid management system 300 such as, for example system fluid pressure, fluid temperature, or inflow pump speed (e.g., rpm) which may correlate to flow rate. The user may also configure parameters and alarms (such as, but not limited to, a system pressure limit, an inflow pump speed limit, etc.), information to be displayed, etc. The touch screen interface may allow the user to add, change, and/or discontinue the use of various modular systems within the fluid management system 300. The touch screen interface may also be used to change the fluid management system 300 between automatic and manual modes for various procedures. It is contemplated that other systems configured to receive user input may be used in place of or in addition to the touch screen interface.

In some embodiments, the touch screen interface may be configured to include selectable areas like buttons and/or may provide a functionality similar to physical buttons as would be understood by those skilled in the art. The display may be configured to show icons related to modular systems and devices included in the fluid management system 300. In some embodiments, the display may include an estimated flow rate display. The estimated flow rate display may be determined based on based on the current size of the electronically adjustable orifice 330, the first fluid pressure measured by the first pressure sensor 350, and the second fluid pressure measured by the second pressure sensor 360, and/or other known values or characteristics.

In some embodiments, the operating parameters may be adjusted by touching a corresponding portion of the touch screen interface. The touch screen interface and/or the display may also display visual alerts and/or issue audio alarms if parameters (e.g., pump speed, system pressure, fluid temperature, etc.) are above or below predetermined thresholds and/or ranges. The touch screen interface and/or the display may also be configured to display any other information the user may find useful during the procedure. In some embodiments, the fluid management system 300 may also include further user interface components such as a heater user interface, a fluid control interface, or other device to manually control various modular systems.

The touch screen interface may be operatively connected to or may be a part of the controller 320. The controller 320 may be a computer, tablet computer, or other processing device. The controller 320 may be operatively connected to one or more system components such as, for example, the inflow pump 310, the fluid warming system 322, a fluid deficit management system, etc. In some embodiments, these features may be integrated into a single unit. The controller 320 is capable of and configured to perform various functions such as calculation, control, computation, display, etc. The controller 320 is also capable of tracking and storing data pertaining to the operations of the fluid management system 300 and each component thereof. In an illustrative embodiment, the controller 320 includes wired and/or wireless network communication capabilities, such as ethernet or Wi-Fi, through which the controller 320 may be connected to, for example, a local area network. The controller 320 may also receive signals from one or more of the sensors of the fluid management system 300, the connected medical device (e.g., the endoscope 100, etc.), the electronically adjustable orifice 330, and/or the electronically adjustable orifice assembly. In some embodiments, the controller 320 may communicate with databases for best practice suggestions and the maintenance of patient records which may be displayed to the user on the display.

In some embodiments, the inflow pump 310 may be a peristaltic pump. In some embodiments, the inflow pump 310 may include multiple pumps or more than one pump. The inflow pump 310 may be electrically driven and may receive power from a line source such as a wall outlet, an external or internal electrical storage device such as a disposable or rechargeable battery, and/or an internal power supply. The inflow pump 310 may operate at any desired speed sufficient to deliver fluid at a target system pressure and/or at an estimated fluid flow rate. In some embodiments, the controller 320 may be configured to automatically adjust one or more outputs for controlling the inflow pump 310.

In some embodiments, the one or more outputs for controlling the inflow pump 310 may also be manually adjusted via, for example, the touch screen interface or a separate fluid controller. While not explicitly shown, the controller 320 may include a separate user interface including buttons that allow the user to increase or decrease the speed and/or the output of the inflow pump 310. In some embodiments, the fluid management system 300 may include multiple pumps having different flow capabilities. Since parameters and/or characteristics of the fluid management system 300 are generally known in advance, inflow pump speed may be correlated to flow rate within the fluid management system 300. In addition or alternatively, in some embodiments, the fluid management system 300 may optionally include a flow rate sensor to measure actual fluid flow rate. The flow rate sensor may be operably connected to the controller 320 and data from the flow rate sensor may be used by the controller 320 to change selected system parameters.

Inflow pump speed, estimated fluid flow rate, actual fluid flow rate, and/or system pressure at any given time may be displayed on the display to allow the operating room (OR) visibility for any changes. If the OR personnel notice a change in inflow pump speed, estimated fluid flow rate, actual fluid flow rate, and/or system pressure that is either too high or too low, the user may manually adjust one or more outputs for controlling the inflow pump 310 and/or the inflow pump speed, estimated fluid flow rate, actual fluid flow rate, and/or system pressure, back to a preferred level. In some embodiments, the fluid management system 300 and/or the controller 320 may monitor and automatically adjust one or more outputs for controlling the inflow pump 310.

In some embodiments, the fluid management system 300 may optionally include the fluid warming system 322 for heating fluid to be delivered to the patient and/or the treatment site. The fluid warming system 322 may include a heater and a heater cassette. The heater cassette may be configured to be a single use heater cassette while the heater may be reused for multiple procedures. For example, the heater cassette may isolate fluid flow such that the heater may be reused with minimal maintenance. The heater cassette may be formed of, for example, polycarbonate or any high heat rated biocompatible plastic and is formed as a single unitary and/monolithic piece or a plurality of pieces permanently bonded to one another. In some embodiments, the heater cassette may include a fluid inlet port and a fluid outlet port located at a lateral side of the heater cassette. The fluid inlet port and the fluid outlet port may each be configured to couple to the supply line(s) of the fluid management system 300. For example, the fluid inlet port may couple the heater cassette and/or the fluid warming system 322 to the fluid source 314 (via the inflow pump 310) using the supply line(s) and/or the fluid source line 312, while the fluid outlet port may couple the fluid warming system 322 with a connected medical device (e.g., the endoscope 100, etc.) via the fluid inflow line 316.

In some embodiments, the heater cassette may include an internal flow path along a channel through which fluid may flow from the fluid inlet port to the fluid outlet port. The heater cassette, the channel, and/or the internal flow path may include one fluid flow path or multiple fluid flow paths. In some embodiments, the channel may pass through a susceptor which may allow the fluid to be heated via induction heating. When the heater cassette is coupled with the heater, the susceptor may be configured to be positioned within an induction coil. Other fluid warming system configurations and methods may also be used, as desired. For example, the heater may include one or more heat sources such as, for example a platen system or an inline coil in the supply line(s) using electrical energy. Heating may be specifically designed and tailored to the inflow pump speed, fluid flow rates, and/or system pressure required in the specific application of the fluid management system 300. Some illustrative fluid warming systems are described in described in commonly assigned U.S. Patent Application Publication No. 2018/0361055, titled AUTOMATED FLUID MANAGEMENT SYSTEM, the entire disclosure of which is hereby incorporated by reference.

In some embodiments, the fluid warming system 322 may include a heater user interface separate from the touch screen interface. The heater user interface may simply be a display screen providing a digital display of the internal temperature of the heater. In another embodiment, the user interface, the controls 340, and/or the connected medical device (e.g., the endoscope 100, etc.) may also include temperature adjustment buttons to increase or decrease the temperature of the heater. In some embodiments, the heater user interface and/or the display screen may indicate the current temperature of the heater as well as the target temperature to be reached. It is noted that all information output from the fluid warming system 322 may be transmitted directly to the display such that no heater user interface is necessary.

The fluid warming system 322 may include one or more sensors configured to monitor the fluid flowing therethrough. For example, temperature sensors may be mounted in the fluid warming system 322 such that they detect the temperature of the fluid flowing through the heater cassette. The temperature sensors may be located at or near the fluid inlet port and/or the fluid outlet port. In some embodiments, the temperature sensors may be mounted so that they detect the temperature of fluid flowing through the heater cassette prior to the fluid entering the susceptor and after fluid exits the susceptor. In some embodiments, additional sensors may be located at a medial portion of the susceptor so that they detect a progression of temperature increase of the fluid in the heater cassette. The temperature sensors may remotely send any information to the display or they may send information to heater user interface and/or the display screen thereof, if so provided. In another embodiment, the temperature sensors may be hardwired with the heater user interface (if provided) which is then able to remotely transmit desired information to the display. Alternatively, or additionally, the temperature sensors may be hardwired to and/or with the controller 320.

The heater may further include at least one pressure sensor configured to monitor system pressure and/or a bubble sensor configured to monitor the fluid flowing through the system for bubbles. The heater cassette may include a corresponding pressure sensor interface and bubble sensor interface that allow the at least one pressure sensor and the bubble sensor, respectively, to monitor the fluid flowing through the heater cassette when the heater cassette is coupled with the fluid warming system 322. The at least one pressure sensor and/or the bubble sensor may remotely and/or electronically send data and/or information to the controller 320, to the display, and/or to the heater user interface and/or the display screen thereof, if so provided. The controller 320 may be configured to receive pressure signals from the at least one pressure sensor, the pressure signals corresponding to a system pressure within the fluid management system 300. In some embodiments, the at least one pressure sensor and/or the bubble sensor may be hardwired with the heater user interface (if provided) which is then able to remotely transmit desired information to the display. Alternatively, or additionally, the at least one pressure sensor and/or the bubble sensor may be hardwired to and/or with the controller 320.

In some embodiments, the at least one pressure sensor may include two pressure sensors, three pressure sensors, or more pressure sensors. In some embodiments having two or more pressure sensors, the individual pressure sensors may be spaced apart from each other. In some embodiments, the at least one pressure sensor may be positioned downstream of the inflow pump 310. In some embodiments, the at least one pressure sensor may be positioned upstream of the fluid inflow line 316. In some embodiments, the at least one pressure sensor may be positioned downstream of the inflow pump 310 and upstream of the fluid inflow line 316. In some embodiments, the at least one pressure sensor may be configured to detect the system pressure within the fluid management system 300 downstream of the inflow pump 310 and/or upstream of the fluid inflow line 316.

In some embodiments, the heater cassette may collectively act as a fluid reservoir. In some embodiments, the fluid reservoir of the heater cassette may include a pulsation dampener to reduce peristaltic pulsations, and one or more air traps to remove bubbles before and/or after heating the fluid flowing through the heater cassette. In some embodiments, the pulsation dampener and the one or more air traps may collectively act as the fluid reservoir. Fluid level(s) within the fluid reservoir of the heater cassette may rise and fall based on a ratio between an inflow amount of fluid being pumped into the heater cassette and an outflow amount of fluid exiting the heater cassette.

In each configuration, the fluid management system 300 may operate in one or more different modes — for example, a “pressure control mode”, a “flow compensation mode”, etc. In the pressure control mode, the controller 320 may modulate various system parameters and/or the one or more outputs to the inflow pump 310 to keep and/or maintain the system pressure at a system pressure set point, which may be entered by the user on the touch screen interface. In some embodiments, the system pressure set point may be set and/or selected automatically based on which type and/or configuration of connected medical device (e.g., the endoscope 100, etc.) is fluidly connected to the inflow pump 310 and/or the fluid management system 300. As discussed herein, the system pressure may be measured by the at least one pressure sensor within the fluid management system 300.

In some embodiments, the fluid management system 300 may be configured to be fluidly connected to a working lumen of a medical device (e.g., an endoscope 100). As such, the fluid management system 300 may be configured to control an inflow of fluid from the fluid management system 300 through the medical device (e.g., the endoscope 100) to the treatment site. In at least some embodiments, the working lumen of the medical device (e.g., the endoscope 100) may also be used to insert a medical instrument or tool through the medical device (e.g., the endoscope 100) to the treatment site. Insertion of the medical instrument or tool may partially obstruct the working lumen and thus affect the flow and/or pressure characteristics of the inflow of fluid. In the flow compensation mode, the fluid management system 300 and/or the controller 320 may be configured to automatically modulate selected system parameters to attempt to maintain a desired or estimated flow rate through the fluid inflow line 316 and/or the medical device (e.g., the endoscope 100). Thus, the fluid management system 300 may be configured to attempt to overcome the partial obstruction of the working lumen. As such, communication with the electronically adjustable orifice 330 and/or the current size of the electronically adjustable orifice 330 may be relevant to operation of the fluid management system 300. Accordingly, the fluid management system 300 and/or the controller 320 may be in electronic communication with the inflow pump 310, the controls 340, the control circuitry, and/or the electronically adjustable orifice 330, as indicated by dashed lines in the figures.

In some embodiments, the control circuitry and/or the controller 320 may be configured to receive a signal of a current size of the electronically adjustable orifice 330 and/or may be configured to send a signal to change a size of the electronically adjustable orifice 330, and thus change a flow rate of fluid passing through the orifice 330. In some embodiments, the control circuitry and/or the controller 320 may be configured to calculate and/or estimate an approximate current flow rate of a fluid passing through the electronically adjustable orifice 330 based on the current size of the electronically adjustable orifice 330 and a system pressure of the fluid measured between the inflow pump 310 and the electronically adjustable orifice 330. In some embodiments, the control circuitry and/or the controller 320 may be configured to calculate and/or estimate an approximate current flow rate of a fluid passing through the electronically adjustable orifice 330 based on the current size of the electronically adjustable orifice 330 and a system pressure of the fluid measured between the inflow pump 310 and the fluid inflow line 316.

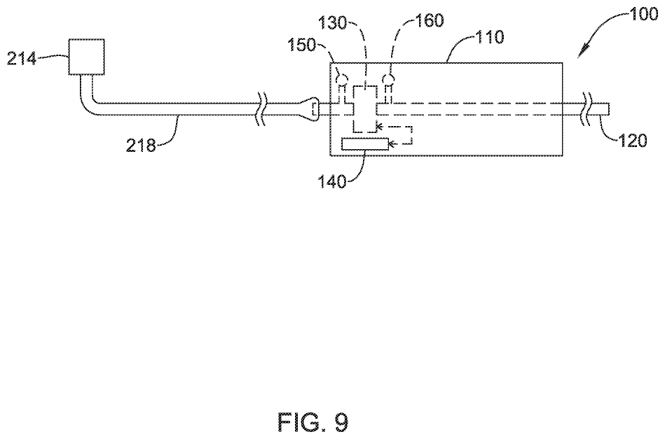

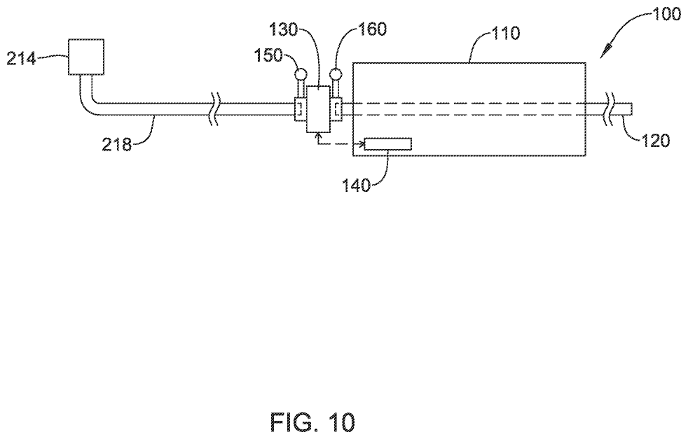

FIGS. 9-10 schematically illustrate selected aspects of an endoscopic system. The endoscopic system may include the endoscope 100 as described herein. In some embodiments, the endoscope 100 may include the electronically adjustable orifice 130 associated with the inflow port of the handle 110. In some embodiments, the electronically adjustable orifice 130 may be disposed within the handle 110, as shown in FIG. 9. In some embodiments, the electronically adjustable orifice 130 may be disposed outside of the handle 110, as shown in FIG. 10. In some embodiments, the electronically adjustable orifice 130 may be integrated into the handle 110. In some embodiments, the electronically adjustable orifice 130 may be a separate component that is attachable to the inflow port of the handle 110. In some embodiments, the electronically adjustable orifice 130 may include one or more motors, gears, cams, pulleys, etc. configured to and/or capable of managing, maintaining, and/or changing a size of the electronically adjustable orifice 130.

The endoscopic system may include controls 140 associated with the handle 110. In some embodiments, the controls 140 may be integrally formed in the handle 110. In some embodiments, the controls 140 may be fixedly secured to an outer surface of the handle 110. In some embodiments, the controls 140 may be a separate element that may be added on to the endoscope 100 and/or the handle 110. In some embodiments, the controls 140 may be removably secured to the outer surface of the handle 110. Other configurations are also contemplated. The endoscopic system may include control circuitry for receiving a signal of a current size of the electronically adjustable orifice 130 and/or for sending a signal to change a size of the electronically adjustable orifice 130. In at least some embodiments, the control circuitry may be associated with the controls 140. In some embodiments, the control circuitry may be disposed within the handle 110 of the endoscope 100. In some embodiments, the control circuitry may be disposed outside of the handle 110 of the endoscope 100. In some embodiments, the control circuitry may be in electronic communication with the controls 140. In some embodiments, the controls 140 and/or the control circuitry may be in electronic communication with the electronically adjustable orifice 130, as indicated by a dashed line between these elements in the figures. In some embodiments, the controls 140 and/or the control circuitry may be hardwired to the electronically adjustable orifice 130. In some embodiments, the controls 140 and/or the control circuitry may be wirelessly connected to and/or in wireless communication with the electronically adjustable orifice 130. Other configurations are also contemplated.

Accordingly, a user of the endoscope 100 may be able to use the controls 140 to change the size of the electronically adjustable orifice 130 and thus change the flow rate of fluid delivered to a patient's body through the endoscope 100. For example, when the user actuates the controls 140, the control circuitry may send a signal to the electronically adjustable orifice 130 to change the size of the electronically adjustable orifice 130. In some embodiments, the controls 140 may include one or more of speed control, home position, fully open, fully closed, increase size, decrease size, etc. In some embodiments, the controls 140 may include buttons, dials, knobs, slides, touch interface(s), voice interface(s), etc. In some embodiments, the controls 140 may include multiple different types of controls (e.g., buttons and knobs, etc.).

In some embodiments, the endoscopic system may include a first pressure sensor 150 disposed upstream of the electronically adjustable orifice 130. In some embodiments, the first pressure sensor 150 may be disposed immediately upstream of the electronically adjustable orifice 130. In some embodiments, the endoscopic system may include a second pressure sensor 160 disposed downstream of the electronically adjustable orifice 130 and upstream of the elongate shaft 120 of the endoscope 100. In some embodiments, the second pressure sensor 160 may be disposed immediately downstream of the electronically adjustable orifice 130 and upstream of the elongate shaft 120 of the endoscope 100.

In some embodiments, the first pressure sensor 150 and/or the second pressure sensor 160 may be integrated into the handle 110 of the endoscope 100. In some embodiments, the first pressure sensor 150 and/or the second pressure sensor 160 may be integrated into the electronically adjustable orifice 130 to form an electronically adjustable orifice assembly. In some embodiments, the first pressure sensor 150 and/or the second pressure sensor 160 may be standalone elements added to and/or connected to the endoscope 100 and/or the electronically adjustable orifice 130. In some embodiments, the first pressure sensor 150 and/or the second pressure sensor 160 may be releasably connected to the endoscope 100 and/or the electronically adjustable orifice 130.

In some embodiments, the first pressure sensor 150 and/or the second pressure sensor 160 may be disposed downstream of the inflow port, as seen in FIG. 9. In some embodiments, the electronically adjustable orifice assembly may be disposed downstream of the inflow port. In some embodiments, the first pressure sensor 150 and/or the second pressure sensor 160 may be disposed upstream of the inflow port, as seen in FIG. 10. In some embodiments, the electronically adjustable orifice assembly may be disposed upstream of the inflow port. Other configurations are also contemplated.

The first pressure sensor 150 may be configured to measure a first fluid pressure of fluid flowing through the endoscope 100 upstream of the electronically adjustable orifice 130. Preferably, the first pressure sensor 150 may be configured to measure a first fluid pressure of fluid flowing into the electronically adjustable orifice 130 immediately before the fluid enters the electronically adjustable orifice 130. The second pressure sensor 160 may be configured to measure a second fluid pressure of fluid flowing through the endoscope 100 downstream of the electronically adjustable orifice 130. Preferably, the second pressure sensor 160 may be configured to measure a second fluid pressure of fluid flowing out of the electronically adjustable orifice 130 immediately after the fluid exits or leaves the electronically adjustable orifice 130.

Location of the first pressure sensor 150 and the second pressure sensor 160 relative to the electronically adjustable orifice 130 may be pertinent to operation of the endoscopic system and/or the electronically adjustable orifice 130. For example, by locating the first pressure sensor 150 and the second pressure sensor 160 physically close to the electronically adjustable orifice 130 (e.g., within about 5-50 millimeters), the control circuitry may be configured to calculate and/or estimate an approximate current flow rate of the fluid passing through the electronically adjustable orifice 130 based on the current size of the electronically adjustable orifice 130, the first fluid pressure measured by the first pressure sensor 150, and the second fluid pressure measured by the second pressure sensor 160. Fluid flow rate may be calculated and/or estimated based on a combination of known and measured characteristics, as is known in the art. In some embodiments, the first pressure sensor 150 and/or the second pressure sensor 160 may send pressure signals to the control circuitry to facilitate and/or trigger changes in state or system behavior with instantaneous or near-instantaneous response.

The endoscopic system of FIGS. 9-10 may include a “dumb” fluid management system. In some embodiments, the fluid management system may include the fluid source 214 and a fluid line 218 fluidly directly connecting the fluid source 214 to the endoscope 100 and/or the inflow port of the endoscope 100, as seen in FIG. 9. In some embodiments, the fluid management system may include the fluid source 214 and the fluid line 218 fluidly directly connecting the fluid source 214 to the endoscope 100, the electronically adjustable orifice 130, and/or the electronically adjustable orifice assembly, as seen in FIG. 10. The fluid source 214 of the “dumb” fluid management system may be a bag or other reservoir positioned above the endoscope 100, such as on a pole, to facilitate gravity feed of fluid from the fluid source 214 to the endoscope 100. In some embodiments, the entire endoscopic system may be a single use device and/or may be disposable.

As discussed herein, the control circuitry may be configured to calculate and/or estimate an approximate current flow rate of the fluid passing through the electronically adjustable orifice 130 based on the current size of the electronically adjustable orifice 130, the first fluid pressure measured by the first pressure sensor 150, and the second fluid pressure measured by the second pressure sensor 160. In at least some embodiments, the control circuitry may be configured to adjust, modify, and/or control a size of the electronically adjustable orifice 130 to attempt to maintain a generally constant flow rate through the electronically adjustable orifice 130. For example, the control circuitry may be configured to slowly and/or automatically increase the size of the electronically adjustable orifice 130 as the fluid source 214 empties in order to maintain a generally constant flow rate of fluid through the electronically adjustable orifice 130. Other configurations are also possible.

In some alternative embodiments, the “dumb” fluid management system of FIGS. 9-10 may include a constant speed inflow pump (not shown) disposed along the fluid line 218 between the fluid source 214 and the endoscope 100. The constant speed inflow pump may be configured to operate at a fixed pump speed in an on or off configuration, and/or may be devoid of a controller for managing pump speed, etc. Accordingly, the user may activate the controls 140 at and/or on the handle 110 to change the size of the electronically adjustable orifice 130 in order to change the flow rate through the electronically adjustable orifice 130. In some embodiments, the entire endoscopic system may be a single use device and/or may be disposable.

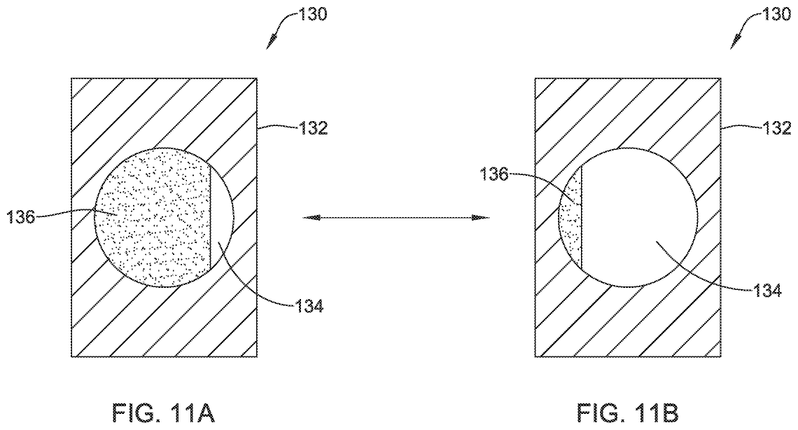

FIGS. 11A-11B schematically illustrate an example electronically adjustable orifice 130 in partial cross-section. As shown, the electronically adjustable orifice 130 may include a housing 132 and a movable restrictor 136 disposed therein. The movable restrictor 136 may be configured to move relative to the housing 132 to change a size of an opening 134 through the electronically adjustable orifice 130. In some embodiments, the movable restrictor 136 may slide laterally and/or horizontally. In some embodiments, the movable restrictor 136 may slide vertically. In some embodiments, the movable restrictor 136 may rotate relative to the opening 134. Other configurations are also contemplated. FIG. 11A illustrates a relatively small opening 134, whereas FIG. 11B illustrates a relatively large opening 134 after the movable restrictor 136 has been moved relative to the housing 132 to increase the size of the opening 134 through the electronically adjustable orifice 130.

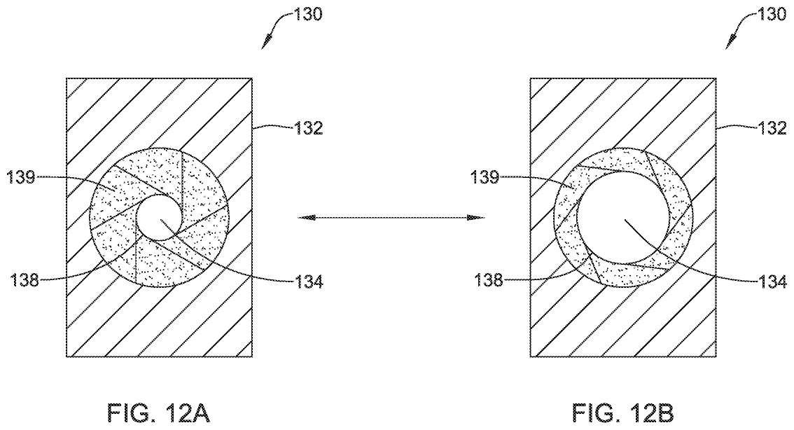

FIGS. 12A-12B schematically illustrate another example electronically adjustable orifice 130 in partial cross-section. As shown, the electronically adjustable orifice 130 may include a housing 132 and an adjustable iris 138 having a plurality of movable leaves 139 arranged around and/or defining a central opening 134. The plurality of movable leaves 139 may be configured to move relative to the housing 132 and/or each other to adjust a size of the adjustable iris 138 and/or the central opening 134. FIG. 12A illustrates a relatively small iris 138 and/or central opening 134, whereas FIG. 12B illustrates a relatively large iris 138 and/or central opening 134 after the plurality of movable leaves 139 has been moved relative to the housing 132 and/or each other to increase the size of the adjustable iris 138 and/or central opening 134 through the electronically adjustable orifice 130.

It shall be understood that the example electronically adjustable orifice(s) 130 of FIGS. 11A-12B is/are merely exemplary. Other configurations are also contemplated.

Those skilled in the art will recognize that the present disclosure may be manifested in a variety of forms other than the specific embodiments described and contemplated herein. Accordingly, departure in form and detail may be made without departing from the scope and spirit of the present disclosure as described in the appended claims.

The materials that can be used for the various components of the system(s) and the various elements thereof disclosed herein may include those commonly associated with medical devices. For simplicity purposes, the following discussion refers to the system. However, this is not intended to limit the devices and methods described herein, as the discussion may be applied to other elements, members, components, or devices disclosed herein, such as, but not limited to, the fluid management system, the endoscopic system, the endoscope, the elongate shaft, the inflow pump, the controller, the supply line(s), the handle, the workstation, the fluid supply source, the electronically adjustable orifice, the pressure sensor(s), and/or elements or components thereof.

In some embodiments, the system, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400,

NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MM) compatibility is imparted into the system and/or other elements disclosed herein. For example, the system and/or components or portions thereof may be made of a material that does not substantially distort the image and create substantial artifacts (i.e., gaps in the image). Certain ferromagnetic materials, for example, may not be suitable because they may create artifacts in an MRI image. The system, or portions thereof may also be made from a material that the MM machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An endoscopic system, comprising:
an endoscope including a handle and an elongate shaft extending distally from the handle;
wherein the handle includes an inflow port in fluid communication with the elongate shaft, the inflow port being configured to fluidly connect to a fluid inflow line;
an electronically adjustable orifice associated with the inflow port; and
control circuitry for receiving a signal of a current size of the electronically adjustable orifice and/or for sending a signal to change a size of the electronically adjustable orifice.

2. The endoscopic system of claim 1, further comprising a first pressure sensor disposed upstream of the electronically adjustable orifice and a second pressure sensor disposed downstream of the electronically adjustable orifice and upstream of the elongate shaft.

3. The endoscopic system of claim 2, wherein the control circuity is configured to calculate an approximate current flow rate of a fluid passing through the electronically adjustable orifice based on the current size of the electronically adjustable orifice, a first fluid pressure measured by the first pressure sensor, and a second fluid pressure measured by the second pressure sensor.

4. The endoscopic system of claim 1, further comprising an inflow pump configured to pump a fluid through the fluid inflow line to the inflow port, wherein the control circuitry is configured to calculate an approximate current flow rate of a fluid passing through the electronically adjustable orifice based on the current size of the electronically adjustable orifice and a system pressure of the fluid measured between the inflow pump and the electronically adjustable orifice.

5. The endoscopic system of claim 1, wherein the electronically adjustable orifice includes an adjustable iris having a plurality of movable leaves arranged around a central opening, the plurality of movable leaves is configured to move to adjust a size of the central opening.

6. The endoscopic system of claim 1, wherein the electronically adjustable orifice is disposed within the handle.

7. The endoscopic system of claim 1, wherein the electronically adjustable orifice is disposed outside of the handle.

8. The endoscopic system of claim 1, wherein the control circuitry is in electronic communication with the electronically adjustable orifice.

9. A surgical fluid management system, comprising:
an inflow pump;
a fluid source line for fluidly connecting the inflow pump to a fluid source;
a fluid inflow line extending downstream from the inflow pump, the fluid inflow line configured to be fluidly connected to an inflow port of a medical device;
a controller configured to control the inflow pump;
an electronically adjustable orifice located along the fluid inflow line; and
control circuitry for receiving a signal of a current size of the electronically adjustable orifice and/or for sending a signal to change a size of the electronically adjustable orifice;
wherein the controller is in electronic communication with the inflow pump and the electronically adjustable orifice.

10. The surgical fluid management system of claim 9, further comprising a first pressure sensor disposed upstream of the electronically adjustable orifice and a second pressure sensor disposed downstream of the electronically adjustable orifice.

11. The surgical fluid management system of claim 10, wherein the control circuity is configured to calculate an approximate current flow rate of a fluid passing through the electronically adjustable orifice based on the current size of the electronically adjustable orifice, a first fluid pressure measured by the first pressure sensor, and a second fluid pressure measured by the second pressure sensor.

12. The surgical fluid management system of claim 9, wherein the control circuitry is configured to calculate an approximate current flow rate of a fluid passing through the electronically adjustable orifice based on the current size of the electronically adjustable orifice and a system pressure of the fluid measured between the inflow pump and the electronically adjustable orifice.

13. The surgical fluid management system of claim 9, wherein the electronically adjustable orifice includes an adjustable iris having a plurality of movable leaves arranged around a central opening, the plurality of movable leaves is configured to move to adjust a size of the central opening.

14. An endoscopic system, comprising:
a fluid management system including a fluid source, an inflow pump, a fluid source line fluidly connecting the fluid source to the inflow pump, a fluid inflow line extending downstream from the inflow pump, and a controller for controlling the inflow pump;
an endoscope including a handle and an elongate shaft extending distally from the handle;
wherein the handle includes an inflow port in fluid communication with the elongate shaft, the inflow port being fluidly connectable to the fluid inflow line;
an electronically adjustable orifice associated with the inflow port;
a first pressure sensor disposed upstream of the electronically adjustable orifice;
a second pressure sensor disposed downstream of the electronically adjustable orifice and upstream of the elongate shaft; and
control circuitry for changing a size of the electronically adjustable orifice;
wherein the controller is in electronic communication with the inflow pump and the electronically adjustable orifice.

15. The endoscopic system of claim 14, wherein the control circuitry is configured to receive a signal of a current size of the electronically adjustable orifice.

16. The endoscopic system of claim 15, wherein the control circuity is configured to calculate an approximate current flow rate of a fluid passing through the electronically adjustable orifice based on the current size of the electronically adjustable orifice, a first fluid pressure measured by the first pressure sensor, and a second fluid pressure measured by the second pressure sensor.

17. The endoscopic system of claim 14, wherein the electronically adjustable orifice includes an adjustable iris having a plurality of movable leaves arranged around a central opening, the plurality of movable leaves is configured to move to adjust a size of the central opening.

18. The endoscopic system of claim 14, wherein the electronically adjustable orifice is disposed within the handle.

19. The endoscopic system of claim 14, wherein the electronically adjustable orifice is disposed outside of the handle.

20. The endoscopic system of claim 14, wherein the control circuitry is disposed within the handle.
